(19) United States
(12) Patent Application Publication
(10) Pub. No.: US 2023/0270333 A1
(43) Pub. Date: Aug. 31, 2023

(54) 2-PHOTON ENDOSCOPIC FLUORESCENCE IMAGING PROBE WITH MULTIPLE, BENT, SLANTED-CUT COLLECTION FIBERS

(71) Applicant: Board of Regents, The University of Texas System, Austin, TX (US)

(72) Inventors: Adela Ben-Yakar, Austin, TX (US); Kaushik Subramanian, Austin, TX (US); Ilan Gabay, Austin, TX (US); Liam Andrus, Austin, TX (US); Berk Camli, Austin, TX (US)

(21) Appl. No.: 18/158,237

(22) Filed: Jan. 23, 2023

Related U.S. Application Data
(60) Provisional application No. 63/301,723, filed on Jan. 21, 2022.

(51) Int. Cl. A61B 5/00

(52) U.S. Cl. CPC A61B 5/0062; A61B 5/0082; A61B 1/00096

(57) ABSTRACT

Imaging components and systems are described herein. An example imaging component can include: a housing; at least one excitation optical element at least partially disposed within the housing; at least one laser-guiding element at least partially disposed within the housing, the at least one laser-guiding element being configured to deliver excitation pulses to a target location through the at least one excitation optical element via an aperture; and a signal collecting element disposed adjacent to the at least one excitation optical element.

### CROSS-REFERENCE TO RELATED APPLICATIONS

This application claims the benefit of U.S. provisional patent application No. 63/301,723, filed on Jan. 21, 2022, and titled “TWO-PHOTON ENDOSCOPIC FLUORESCENCE IMAGING PROBE WITH MULTIPLE, BENT, SLANTED-CUT COLLECTION FIBERS,” the disclosure of which is expressly incorporated herein by reference in its entirety.

### STATEMENT REGARDING FEDERALLY FUNDED RESEARCH

This invention was made with government support under Grant numbers R01 EB030061 and R01 DC014783 awarded by the National Institutes of Health. The government has certain rights in the invention.

### BACKGROUND

Imaging devices (e.g., probes) may be used to investigate living tissue. including, for example, living cells. Many imaging devices are plagued by technical challenges and limitations. For example, many imaging devices are not sensitive or specific enough for medical applications including early cancer detection.

Diagnosis of cancer in early stages highly reduces mortality and morbidity. Micro-scale metabolic changes in tissue due to pre-cancer development often precede morphological changes. Therefore, imaging modalities capable of detecting functional changes over small areas can increase sensitivity and specificity of early cancer detection. Label-free imaging of metabolic activity at cellular level resolution, over full thickness of cervix epithelium is possible with two-photon (2p) imaging. However, low probability of 2p excitation and scattering nature of tissues limit autofluorescence levels in 2p imaging.

Embodiments of the present disclosure provide a 2p autofluorescence endoscope system for detection of metabolic changes in cervix in a clinical setting, with an alternative autofluorescence collection method with increased collection efficiency in scattering media. In some embodiments, collection of autofluorescence signals is done with a multitude of high Numerical Aperture (NA) fibers arranged around a miniaturized excitation objective. By cleaving the collection fibers at a specific angle, the directivity of the collection and increase collection efficiency per fiber can be increased.

An exemplary imaging probe in accordance with the present disclosure performs imaging at 775 nm, which corresponds to 2p autofluorescence excitation wavelengths of nicotinamide adenine dinucleotide phosphate (NAD(P)H) and flavin adenine dinucleotide (FAD). In some implementations, laser pulses of femtosecond (fs) duration are delivered to a sample with an air core photonic bandgap fiber. The fiber can be scanned in a spiral pattern via a piezo actuator tube. Scanning at different tissue depths is possible with the axial actuation of the probe part of the endoscope with a linear stepper motor. Benchtop tests indicate that the exemplary endoscope system has lateral and axial resolutions of 0.64 μm and 4.10 μm, respectively. Autofluorescence images with a field-of-view (FOV) of 120 μm can be obtained from freshly excised porcine vocal fold tissue samples.

### SUMMARY

Various embodiments described herein relate to methods, apparatuses, and systems for providing an imaging system, such as, for example, an endoscopic probe. An example imaging component can comprise: a housing; at least one excitation optical element at least partially disposed within the housing; at least one excitation optical element at least partially disposed within the housing; the at least one excitation optical element comprising at least one laser-guiding element being configured to deliver excitation pulses to a target location through the at least one excitation optical element via an aperture; and a signal collecting element disposed adjacent to the at least one excitation optical element.

In some implementations, the signal collecting element is configured to receive the emitted and/or scattered nonlinearly generated signals without interacting with the at least one excitation optical element.

In some implementations, the emitted and/or scattered nonlinearly generated signals comprise at least one of two-photon fluorescence signals, three-photon fluorescence signals, second harmonic generation signals, and third harmonic generation signals.

In some implementations, the imaging probe is operatively coupled to a spacer component that is configured to be positioned adjacent to the signal collecting element, and wherein the spacer component is configured to create a space between the signal collecting element and a tissue.

In some implementations, the signal collecting element comprises a plurality of fibers circumferentially arranged around at least a portion of the at least one laser-guiding element.

In some implementations, the plurality of fibers comprises a number between three and forty fibers.

In some implementations, each of the plurality of fibers is cleaved at an angle.

In some implementations, the angle is between 1 degree and 50 degrees.

In some implementations, the signal collecting element comprises multiple rings of fibers.

In some implementations, the laser-guiding element comprises at least one fiber extending through at least a portion of the housing.

In some implementations, the at least one fiber comprises an air-core bandgap fiber or an air-core Kagome fiber.

In some implementations, the aperture comprises a transparent window.

In some implementations, the at least one excitation optical element comprises a plurality of focusing lenses.

In some implementations, the imaging probe further comprises a detector component operatively coupled to the imaging probe that is configured to receive the emitted and/or scattered nonlinearly generated signals and output image data via a display.

In some implementations, wherein the detector component comprises at least one of a photomultiplier-tube (PMT) module and a Hybrid (HyD) detector.

In some implementations, the imaging probe is embodied as an endoscope or table-top nonlinear microscope.

In some embodiments, the imaging probe further comprises a handle portion, wherein the at least one laser-guiding element and at least a portion of the signal collecting element extends through the handle portion and a length of the housing.

In some implementations, the imaging probe further comprises a motor disposed within the handle portion.

In some implementations, the motor is configured to actuate axial scanning of the imaging probe to facilitate an imaging depth adjustment.

In some implementations, a system is provided. The system can comprise: an imaging probe, the imaging probe comprising: a housing, at least one excitation optical element at least partially disposed within the housing, the at least one excitation optical element comprising at least one laser-guiding element being configured to deliver excitation pulses to a target location through the at least one excitation optical element via an aperture, and a signal collecting element adjacent to the at least one excitation optical element; and a detector component operatively coupled to the imaging probe that is configured to receive emitted and/or scattered nonlinearly generated signals via the signal collecting element and output image data via a display.

In some implementation, the signal collecting element is configured to receive the emitted and/or scattered nonlinearly generated signals without interacting with at least one of the at least one excitation optical element while interacting with at least one focusing optical element external to the probe.

In some implementations, the imaging probe has an imaging signal depth between 0-2000 μm.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A is an example perspective view of an imaging probe according to implementations described herein.

FIG. 1B is another example perspective view of an imaging probe according to implementations described herein.

FIG. 2A is an example side view of an imaging probe according to implementations described herein.

FIG. 2B is an example side view of an imaging probe according to implementations described herein.

FIG. 3 is an example section view of an imaging probe according to implementations described herein.

FIG. 4 is another example perspective view of an imaging probe according to implementations described herein.

FIG. 5 is an example side section view of a probe portion according to implementations described herein.

FIG. 6 is an example side section view of a probe portion according to implementations described herein.

FIG. 7 is an example perspective view of a signal collecting element according to implementations described herein.

FIG. 8A and FIG. 8B are schematic diagrams depicting an axial scanning operation in relation to an imaging probe according to implementations described herein.

FIG. 9 is a schematic diagram depicting an example miniaturized objective according to implementations described herein.

FIG. 10A and FIG. 10B are graphs depicting Strehl ratio profiles for on-axis and off-axis focus, respectively.

FIGS. 11A-J are schematic diagrams depicting results of various simulations.

FIG. 12A is a schematic diagram depicting an example collection fiber bundle is provided according to implementations described herein.

FIG. 12B and FIG. 12C are schematic diagrams depicting example collection optics systems according to implementations described herein.

FIG. 13A and FIG. 13B are graphs depicting measured deflection characteristics of the focal spot.

FIGS. 14A-D show results of a Gaussian function was fit to the cross-sections of the intensity map obtained by the beam profiler with R2 of 0.99.

FIGS. 15A-E show images of a pollen core cluster obtained with the imaging probe obtained at different imaging depths/different axial positions.

FIGS. 16A-0 show autofluorescence images obtained with the probe at different imaging depths into a freshly excised porcine vocal fold tissue sample.

FIG. 17 is a block diagram that illustrates an example computing device.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. This disclosure contemplates that the imaging systems, apparatuses and methods described herein can be used in a variety of applications, including fluorescence microscopy, medical imaging (e.g., endoscopy), and/or the like. The imaging systems, apparatuses, and methods can be used to output image data, for example depicting living tissue and/or measurements via a display.

Two-photon excitation fluorescence (TPEF or 2PEF) or microscopy refers to a fluorescence-based imaging technique that facilitates imaging of living tissue. In TPEF, two photons are simultaneously absorbed by an electron, followed by decay via spontaneous emission of a single photon. The probability of excitation is well-confined to a small area, increasing spatial resolution when applied to fluorescence imaging applications. The excitation wavelength is larger than the emission wavelength, enabling excitation within higher tissue depth. For example, TPEF in endoscopic applications allows imaging of tissue in increased depth in areas that are harder to reach with minimal invasion. Additionally, label-free imaging is possible with reliance on autofluorescence.

TPEF requires concurrent excitation of two photons with longer wavelength than the emitted light. For each excitation, two photons of light (e.g., near-infrared (NIR)) are absorbed. In TPEF applications, two exciting photons that each have a lower photon energy value required for a photon excitation are used to excite a fluorophore in a quantum event. The excitation leads to emission of a fluorescence photon with a quantum yield that would result from conventional single-photon absorption at a higher energy (shorter wavelength) than either of the two exciting photons. By using two exciting photons, the resulting axial spread is substantially lower than for single-photon excitation. Accordingly, the extent in the z-dimension is improved such that thin optical sections can be cut. Additionally, the use of lower energy/long wavelengths such as infrared is advantageous in live cell imaging because such wavelengths cause less damage than single-photon excitation techniques. TPEF may be used in a variety of imaging and medical applications including cancer, kidney, and embryonic research and/or investigation.

Many types of cancers can be cured if they can be detected in an early or pre-cancerous stage. The majority of all human cancers form in epithelium, where metabolic changes at cellular level occur long before large-scale morphological transformations appear. However, inspection via low-resolution imaging techniques such as endoscopy, colposcopy, and colonoscopy, followed by biopsy collection is the usual gold standard of cancer diagnosis.

Biopsy collection with the guidance of low-resolution imaging techniques have several drawbacks and is vulnerable to false positive and false negative results. Such visualization techniques allow evaluation of superficial and large-scale morphological changes only. However, large-scale morphological changes associated with cancer development can also be caused by benign conditions. Moreover, since biopsy is a painful, time consuming, and labor-intensive process, only a limited number of samples can be collected.

A recent approach to assist early cancer diagnosis is tissue classification based on quantitative metabolic and morphological parameters such as the rate of certain metabolic activities, mitochondrial organization, and cytoplasm/nucleus ratios of cells. Since pre-cancerous changes can be very subtle and confined, early cancer diagnosis will benefit from assistance of imaging techniques offering metabolic information at high resolution. Magnetic resonance imaging (MRI) and positron emission tomography (PET) imaging provide functional information, but they cannot achieve cellular level resolution desirable in early cancer diagnosis and present additional considerations such as contrast agents and radiation. Optoacoustic imaging, a newer technique, boasts metabolic imaging resolution levels at hundreds of microns. However, the sensitivity and specificity to potentially subtle and localized metabolic changes related to early stages of cancer can be limited with this modality, which relies on hemoglobin, lipids, and water as sources of contrast.

Fluorescence-based imaging (e.g., 2p autofluorescence imaging) has several advantages addressing the challenges of early cancer diagnosis. In this modality, 2p fluorescence excitation relies on simultaneous absorption of two photons. Since the probability of absorption is quadratic, fluorescence becomes confined to an ultrashort laser focal volume, increasing imaging resolution and depth. Imaging with fluorescence is useful since tissues contain endogenous fluorophore molecules naturally. Among them, NAD(P)H and FAD are particularly well-studied as two coenzymes taking part in significant metabolic activities such as glycolysis, which can be indicators of pre-cancerous transformations. The fact that these coenzymes are not present in the nucleus but in cytoplasm and mitochondria, makes them useful for the assessment of mitochondrial organization and cytoplasm/nucleus ratios. As such, 2p autofluorescence imaging has the potential of label-free metabolic imaging at cellular level resolution, over the full thickness of human epithelium.

Whereas benchtop 2p autofluorescence imaging systems have shown the viability of this imaging modality for early detection of cancers using biopsy samples and excised tissues, many clinical applications require in vivo imaging during the examination procedure. Often, it is needed to perform imaging at locations that can be accessed only with probe systems. Therefore, assistance of application specific probes dedicated to selected imaging modality can greatly enhance sensitivity and specificity of early cancer diagnosis. So far, several probes with 2p autofluorescence imaging capability were shown in the literature.

Embodiments of the present disclosure provide a 2p autofluorescence endoscope system intended for clinical assistance in early cancer diagnosis (e.g., cervical cancer). In some implementations, an exemplary probe is configured to operate at an excitation wavelength of 775 nanometers (nm), which corresponds to 2p autofluorescence excitation spectra of NAD(P)H and FAD. Laser pulses of fs duration can be delivered to the sample with an air core photonic bandgap fiber. Focusing of the laser pulses can be handled with a miniaturized objective made of commercially available lenses with 3-millimeter (mm) diameters. The example probe also has the capability to perform axial scanning with a Direct Current (DC) servo motor to be able to obtain images at different depths of the epithelium in a clinical setting. This is significant since recent work in the literature suggests depth dependent information about metabolic and morphological changes may be relevant in classification of healthy and pre-cancerous tissues. Lateral scanning of the focal volume can be performed with a piezo actuator tube in spiral pattern.

Whereas low probability of 2p excitation contributes to high resolution in 2p autofluorescence imaging, it causes the autofluorescence signals to be weaker. In a clinical application, autofluorescence signals will further be weakened by scattering in tissue, particularly at higher imaging depths. Therefore, it is imperative that a high efficiency autofluorescence collection system is implemented in fluorescence-based imaging (e.g., 2p autofluorescence) imaging endoscope systems. Relevant endoscopes presented in the literature so far handle collection mainly in two different ways. One way is to use double clad fibers (DCFs) such that the fiber core is dedicated to excitation and the inner cladding is dedicated to autofluorescence collection. This approach is advantageous in that the collection element is axially aligned with the focal spot. However, available sizes of photonic bandgap fibers limit the overall achievable collection area, potentially limiting the collection efficiency and imaging depth. The second collection approach is directing the autofluorescence signal to another fiber using additional optics. Here, it is possible to use a high diameter multi-modal fiber and increase the collection area. On the other hand, additional optics can introduce additional challenges in optomechanical design and assembly of the endoscope.

Conventional systems generally use a single component (e.g., excitation optics) to emit, focus, and collect a signal, which reduces collection efficiency of such systems. Embodiments of the present disclosure may use separate components for excitation optics and collection optics and thus improve collection efficiency. As an alternative to conventional autofluorescence collection approaches, embodiments of the present disclosure employ a multitude of high numerical aperture (NA) multi-modal fibers arranged around the miniaturized excitation objective. Using a multitude of collection fibers increases the overall collection area significantly. By cleaving these collection fibers at a finite angle, the directivity of the collection and increase collection efficiency per fiber is also increased, particularly at high scattering settings, without the need of directing autofluorescence using additional optical elements for this purpose. Coupling of the output of collection fiber bundle to the sensing a photomultiplier-tube (PMT) can be achieved with dedicated collection optics.

Embodiments of the present disclosure provide an endoscopic probe design for fluorescence-based imaging (e.g., two-photon fluorescence, three-photon fluorescence, second harmonic generation, and third harmonic generation imaging) at adjustable tissue depths. Adjustment of imaging depth is due to axial scanning of probe components via motor actuation. In some implementations, spatial actuation of excitation fiber is handled with a piezo tube. Excitation signals are focused on tissue with an objective fitting the inner diameter of the probe.

Optomechanical Design and Axial Scanning

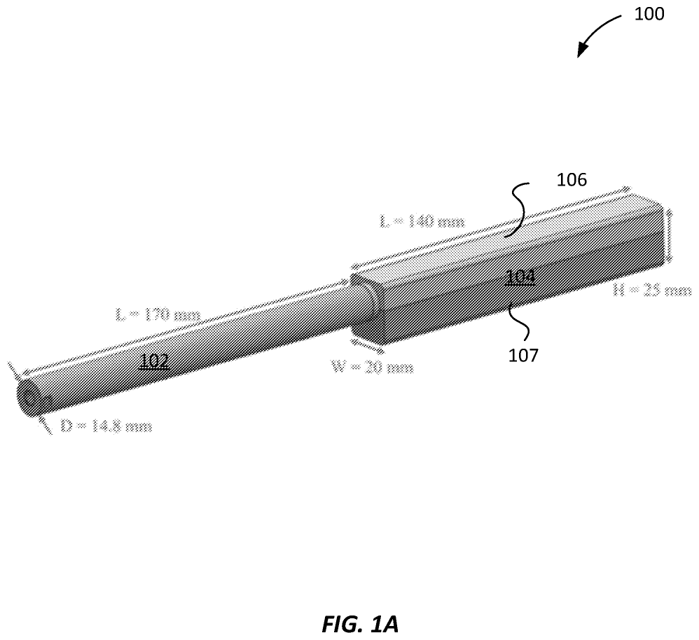

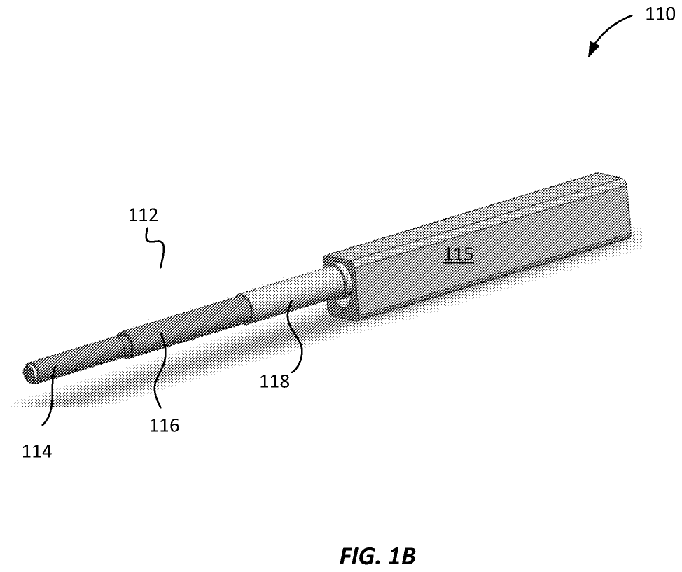

Referring now to FIG. 1A, an example perspective view of an imaging probe 100 (e.g., endoscope) is shown. The imaging probe 100 is configured to deliver excitation pulses to a target location (e.g., tissue) and receive emitted and/or scattered nonlinearly generated signals to facilitate imaging in various applications including endoscopy. As depicted, the imaging probe 100 comprises a probe portion 102 and a handle 104.

In the example shown in FIG. 1A, the probe portion 102 comprises a cylindrical body, and the hand portion 104 comprises a substantially cuboid body. In various examples, the probe portion 102 and the handle 104 may be fixedly attached (e.g., defining a continuous body or housing) or removably attached to one another. As disclosed herein, the imaging probe 100 can comprise at least one excitation optical element (including at least one laser-guiding element and at least one focusing element), at least one signal collecting element, combinations thereof, and/or the like. In some embodiments, each of the noted elements may be at least partially disposed within a housing/body of the imaging probe 100 (e.g., within the probe portion 102 and/or the handle 104).

In various implementations, the handle 104 is configured to house one or more components (e.g., a DC servo motor) and is also suitable for manipulation (e.g., by a clinician conducting an endoscopy). As shown in FIG. 1A, the handle 104 comprises an upper portion 106 and a lower portion 107 that are removably or fixedly attached to one another for easy fabrication and assembly. Additionally, a distal end of the handle 104 is attached (removably or fixedly) to the probe portion 102, which may be an invasive element of an endoscope system.

In various examples, the probe portion 102 can house at least one excitation optical element, including at least one laser-guiding element and at least one focusing optical element, and/or at least one signal collecting element. In some embodiments, an outermost layer of the probe portion 102 comprises or is operatively coupled to a spacer component that is configured to isolate the imaging probe 100 from a target and acts as a spacer at the imaging contact to facilitate axial scanning or z-translation over depth. In some embodiments, a distal end of the probe portion 102 comprises one or more slits (e.g., apertures) that can be left open (e.g., on a distal end of a spacer component, discussed below) to verify axial operation of the imaging probe 100.

Referring now to FIG. 1B, another example perspective view of an imaging probe 110 (e.g., endoscope) is provided. The imaging probe 110 may be similar to the imaging probe 110 described above. Similarly, the imaging probe 110 is configured to deliver excitation pulses to a target location (e.g., tissue) and receive emitted and/or scattered nonlinearly generated signals. As shown, the imaging probe 110 comprises a probe portion 112 and a handle 115.

In the example shown in FIG. 1B, the probe portion 112 comprises a cylindrical body, and the handle 115 comprises a substantially cuboid body. The probe portion 112 and the handle 115 may be fixedly attached or removably attached to one another. The imaging probe 110 can comprise at least one excitation optical element (including at least one laser-emitting element and at least one focusing element), at least one signal collecting element, combinations thereof, and/or the like that are at least partially disposed within the probe portion 112 and/or the handle 115.

In the example shown in FIG. 1B, the probe portion 112 comprises a plurality of casings. In particular, a first casing 114 (e.g., distal casing), a second casing 116 (e.g., intermediary casing), and a third casing 118 (e.g., proximate casing). Each of the first casing 114, the second casing 116, and the third casing 118 comprises a cylindrical member of a different diameter. Additionally, as shown, each of the first casing 114, the second casing 116, and the third casing 118 is configured to be removably received with respect to one another. For example, the first casing 114 is configured to be received within a portion of the second casing 116 and the second casing 116 is configured to be received within a portion of the third casing 118. The first casing 114 defines a distal end of the imaging probe 110 and the third casing 118 is positioned close to/adjacent the handle 115.

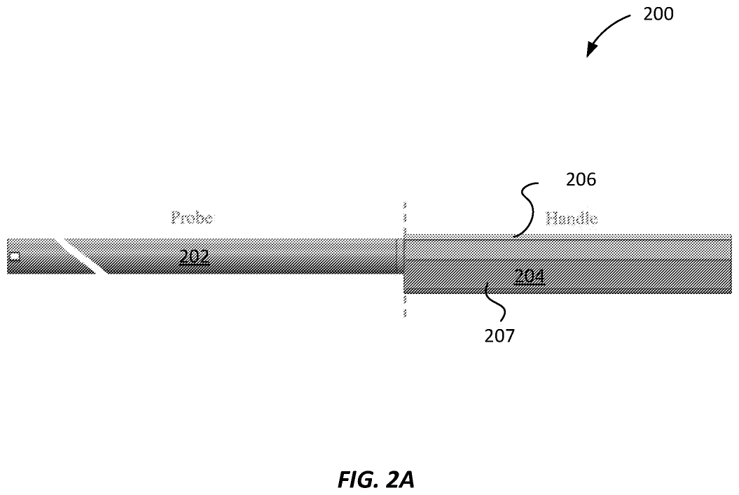

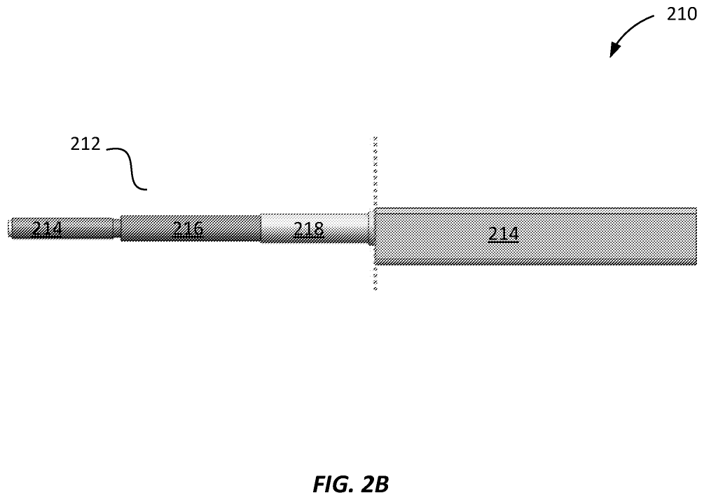

Referring now to FIG. 2A, an example side view of an imaging probe 200 (e.g., endoscope) is shown. The example imaging probe 200 may be similar or identical to the imaging probe 100 described above in connection with FIG. 1A. Similarly, the imaging probe 200 comprises a probe portion 202 and a handle 204. As shown, the probe portion 202 comprises a substantially cylindrical body, and the handle 204 comprises a substantially cuboid body. The imaging probe 200 is configured to house one or more optical components or elements (e.g., at least one excitation optical element including at least one laser-emitting element, at least one signal collecting element, combinations thereof, and/or the like). The probe portion 202 and the handle 204 may be fixedly or removably attached to one another. Additionally, the handle 204 comprises an upper portion 206 and a lower portion 207 that are removably or fixedly attached to one another.

Referring now to FIG. 2B, an example side view of an imaging probe 210 (e.g., endoscope) is provided. The example imaging probe 210 may be similar or identical to the imaging probe 110 described above in connection with FIG. 1B. Similarly, the imaging probe 210 comprises a probe portion 212 and a handle 215. As shown, the probe portion 212 comprises a substantially cylindrical body, and the handle 215 comprises a substantially cuboid body. The imaging probe 210 is configured to house one or more optical components or elements (e.g., at least one excitation optical element, at least one signal collecting element, combinations thereof, and/or the like). The probe portion 212 and the handle 215 may be fixedly or removably attached to one another. As further depicted, the probe portion 212 comprises a plurality of casings (e.g., tubings, members, or the like) that each have different diameters. In the example shown in FIG. 2B, the probe portion 212 comprises a first casing 214 (e.g., distal casing), a second casing 216 (e.g., intermediary casing), and a third casing 218 (e.g., proximate casing). The first casing 214 defines a distal end of the imaging probe 210 and the third casing 218 is positioned close to the handle 215.

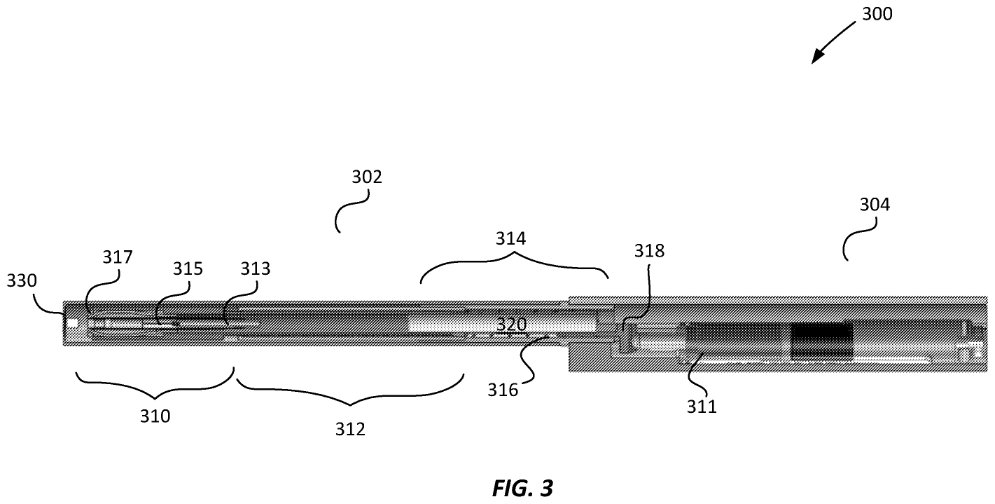

Referring now to FIG. 3, an example section view of an imaging probe 300 (e.g., endoscope) is depicted. The imaging probe 300 may be similar or identical to the imaging probes 100, 200 described above in connection with FIG. 1A and FIG. 2A. As illustrated, the imaging probe 300 comprises a probe portion 302 and a handle 304. In some implementations, the probe portion 302 houses multiple tubing parts. This approach allows the realization of the axial movement and fabrication of the probe portion 302 out of hard material at a length sufficient for cervical imaging.

In the example shown in FIG. 3, the probe portion 302 comprises a first tubing 310 that houses (e.g., contains) various optical elements. The optical elements (e.g., at least one excitation optical element, at least one signal collecting element, or the like) can be configured to slide through a second tubing 312 and a third tubing 314 in a direction that is parallel to a length of the housing of the imaging probe 300. In some embodiments, the second tubing 312 and/or the third tubing 314 are fixedly attached to a surface of the handle 304. In some embodiments, the second tubing 312 comprises a slider part 318 (e.g., ball sliders such as Misumi, BYS8-30) to facilitate sliding without friction. As further depicted in FIG. 3, the imaging probe 300 comprises a spring 316 (e.g., MW Components, 65406S), abutting the second tubing 312 and the slider part 318 on each end and disposed within the third tubing 314. The spring 316 facilitates retraction of the first tubing 310 (e.g., in response to a motor axis retracting). As depicted, the imaging probe 300 comprises a motor 311 disposed within the handle 304. A motor axis of the motor 311 can be in contact with a proximal end of the slider part 318 such that actuation of the motor 311 is translated to the first tubing 310 via a brass tubing 320 connecting the second tubing 312 and the third tubing 314. In some embodiments, the brass tubing 320 is placed inside the spring 316 and thus ensures that the spring 316 retains its axial alignment during axial scanning. A length of the brass tubing can be determined so that the spring 316 has an initial compression even when the first tubing 310 is fully retracted. Since axial actuation compresses the spring 316 further, this neutral compression should be low enough to ensure that the torque on the motor 311 is not excessive when the first tubing 310 is fully actuated.

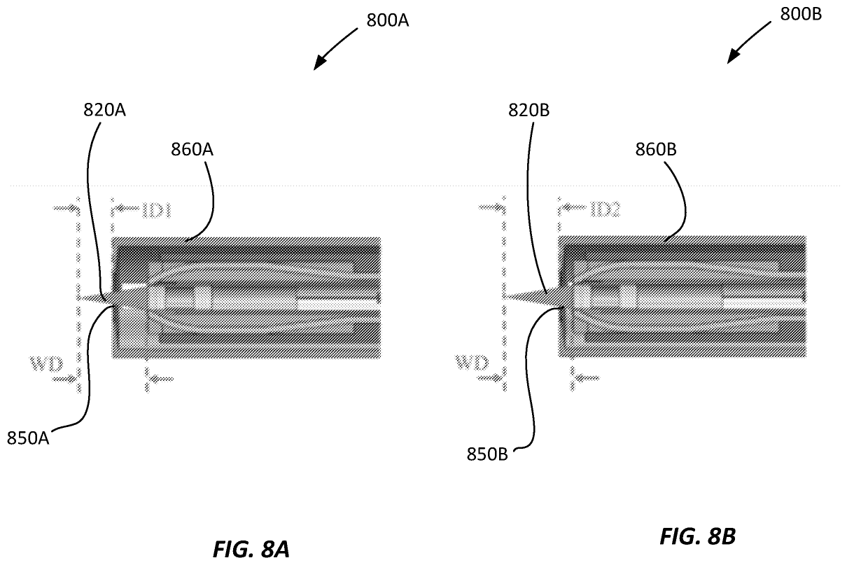

During imaging, axial scanning is used to image at different depths of a given target. In some embodiments, with the distal tip of a spacer component 330 in contact with the target (e.g., tissue), a miniaturized objective present at the distal end of the first tubing 310 is axially scanned as the motor 311 actuates the first tubing 310. This causes the axial scanning of the laser focal spot, since the working distance of the miniaturized objective is fixed. Change of imaging depth with axial scanning is visualized in FIG. 8A and FIG. 8B, detailed below.

As depicted in FIG. 3, the imaging probe 300/excitation optical element further comprises at least one laser-guiding element 315 and at least one actuator 313. In some embodiments, scanning of the at least one laser-guiding element 315 (e.g., excitation fiber) is provided via the at least one actuator 313. By way of example only, the at least one actuator 313 may be a quadrant electrode piezo actuator tube such as, but not limited to, EBL Products, EBL #3, with an outer diameter of 2.36 mm, wall thickness of 0.56 mm, and length of 27.94 mm. In some embodiments, the at least one laser-guiding element 315 comprises a hollow core photonic bandgap fiber (e.g., Thorlabs, HC800B) with a rated mode filed diameter of 5.5 μm and zero-dispersion wavelength of 775 nanometers (nm) used as the excitation fiber. The at least one laser-guiding element 315/excitation fiber can be coaxially aligned and fixed at the distal tip of the at least one actuator 313 (e.g., piezo actuator tube) with an overhang length of 10 millimeters (mm) using an insert piece 317 (e.g., Stereolithography (SLA) printed insert piece). The insert piece 317 can be used to align and anchor the at least one actuator 313 (e.g., piezo actuator tube) within a hypodermic tube with outer diameter of 3.4 mm and wall thickness of 0.15 mm (e.g., MicroGroup, 304H10XX), which also protects the fiber overhang. A short segment of the at least one actuator 313 (e.g., piezo actuator tube) may be exposed at the proximal end so that wire connections can be made to piezo electrodes without the risk of short circuiting to the hypodermic tubing. Wires may be soldered to all quadrant electrodes and to the inner ground electrode. 36 American wire gauge (AWG) gauge wires can be used to keep wire diameters low.

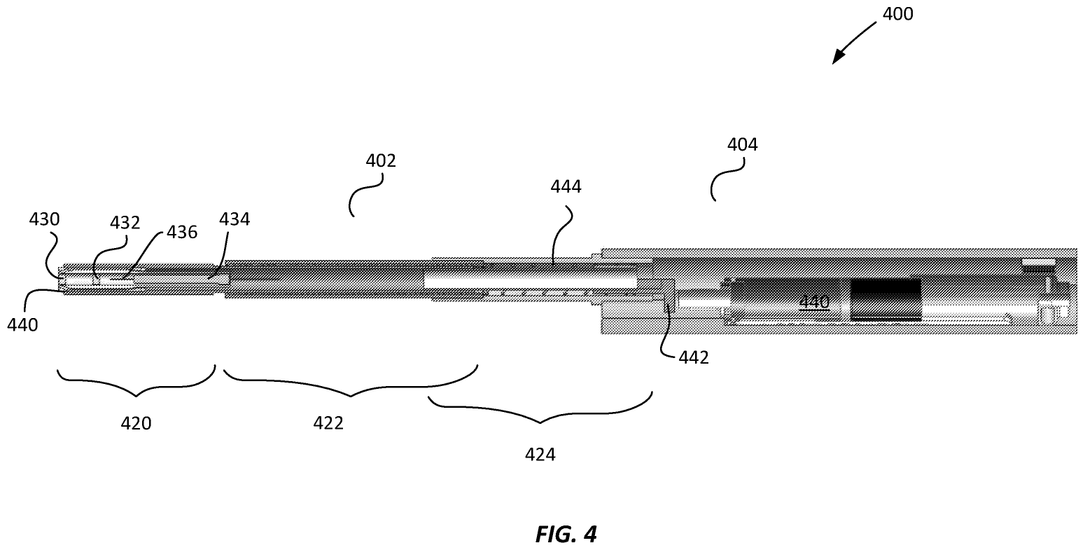

Referring now to FIG. 4, another example perspective view of an imaging probe 400 (e.g., endoscope) is shown. The imaging probe 400 may be similar or identical to the imaging probes 110, 210 discussed above in connection with FIG. 1B and FIG. 2B. The imaging probe 400 is configured to deliver excitation pulses to a target location and receive emitted and/or scattered nonlinearly generated signals in return. As depicted, the imaging probe 400 comprises a probe portion 402 and a handle 404.

As illustrated, the probe portion 402 comprises a substantially cylindrical body, and the handle 404 comprises a substantially cuboid body. As shown, the probe portion 402 and the handle 404 have different lengths (e.g., 160 mm and 142 mm, respectively). In some embodiments, the probe portion 402 comprises a plurality of casings/tubings that each have different diameters. In the example shown in FIG. 4, the probe portion 402 comprises a first casing 420 (e.g., distal casing), a second casing 422 (e.g., intermediary casing), and a third casing 424 (e.g., proximate casing). The first casing 420 defines a distal end of the imaging probe 400 and the third casing 424 is positioned close to the handle 402. In some embodiments, a diameter of the largest casing (the third casing 424) may be 12.7 mm.

As shown, the imaging probe 400 comprises at least one actuator 434, at least one laser-guiding element 436, at least one focusing optical element 430, 432, and at least one signal collecting element 440. Various elements may be at least partially disposed within a housing of the imaging probe 400 (e.g., within the probe portion 402 and/or the handle 404). In some embodiments, the imaging probe 400 may have an imaging signal depth of up to 2000 μm.

As depicted, the imaging probe 400/at least one excitation optical element comprises at least one actuator 434 and at least one laser-guiding element 436 disposed within the housing/body of the probe portion 402. The at least one actuator 434 can comprise a piezo ceramic tube. The at least one laser-guiding element 436 can comprise a photonic bandgap fiber that extends through the handle 404 and a length of the probe portion 402. The at least one laser-guiding element 436 (e.g., photonic bandgap fiber) can be configured to deliver excitation pulses to a target location, such as living tissue, through the at least one excitation optical element (first focusing element 430 and second focusing element 432) via an aperture on a surface of the distal end of the imaging probe 400. In some embodiments, the aperture may be or comprise a transparent window, such as a sapphire window or quartz window. In some implementations, the aperture may be a portion of the spacer component (e.g., cap, cover, tip, or the like).

As illustrated in FIG. 4, the probe portion 402 comprises a first focusing element 430 at a distal end of the probe portion 402 and a second focusing element 432. The first focusing element 430 (e.g., first optical lens) is located a first distance from a distal end of the at least one laser-guiding element 436, and the second focusing element 432 (e.g., second optical lens) is located at a second distance from the distal end of the at least one laser-guiding element 436), where the first distance is greater than the second distance.

The at least one laser-guiding element 436 can be configured to deliver excitation pulses to a target location through the first focusing optical element 430 and the second focusing optical element 432 via an aperture on a distal surface of the probe portion 402 (e.g., adjacent the first focusing optical element 430). In various examples, the at least one laser-guiding element 436 is configured to generate two-photon fluorescence signals, three-photon fluorescence signals, second harmonic generation signals, third harmonic generation signals, and/or the like. The at least one laser-guiding element 436 may be spatially actuated via the at least one actuator 434 (e.g., piezoceramic tube). Excitation pulses can be focused on a target location (e.g., tissue) with an objective fitted on a distal tip (e.g., cap, cover) of the imaging probe 400. The imaging probe 400 can be actuated to image at adjustable depths into tissue (e.g., up to 2000 μm). Depth adjustment can be implemented via axial scanning via a motor 440 disposed within the handle 404. Motor actuation is translated to the probe portion 402 via a slider 442. The slider 442 is configured to move the first casing 420 back and forth (in relation to a length of the imaging probe 400 housing). As depicted, the first casing 420 comprises at least a portion of the at least one excitation optical element 434 and the at least one signal collecting element 440. A linear bearing in the second casing 422 and a spring 444 in the third casing 424 can be utilized to support the actuation mechanism.

Additionally, the imaging probe 400 comprises a signal collecting element 440 (e.g., adjacent to the aperture on a distal end of the probe portion 402). The signal collecting element 440 may be positioned adjacent to a spacer component disposed or positioned on an exterior portion (e.g., rim) of the imaging probe 400. The signal collecting element 440 may comprise a plurality of fibers (e.g., between 3 and 40 distinct fibers) circumferentially arranged around the aperture and/or at least a portion of the at least one laser-guiding element 436. Each of the plurality of fibers can be cleaved at an angle (e.g., between 1 and 50 degrees) to increase collection directivity and efficiency of the imaging probe 400. For example, the signal collecting element 440 may comprise multiple rings of fibers (e.g., at least one air-core bandgap fiber or at least one air-core Kagome fiber). The signal collecting element 440 can be configured to receive emitted and/or scattered nonlinearly generated signals without interacting with the laser-guiding element 436.

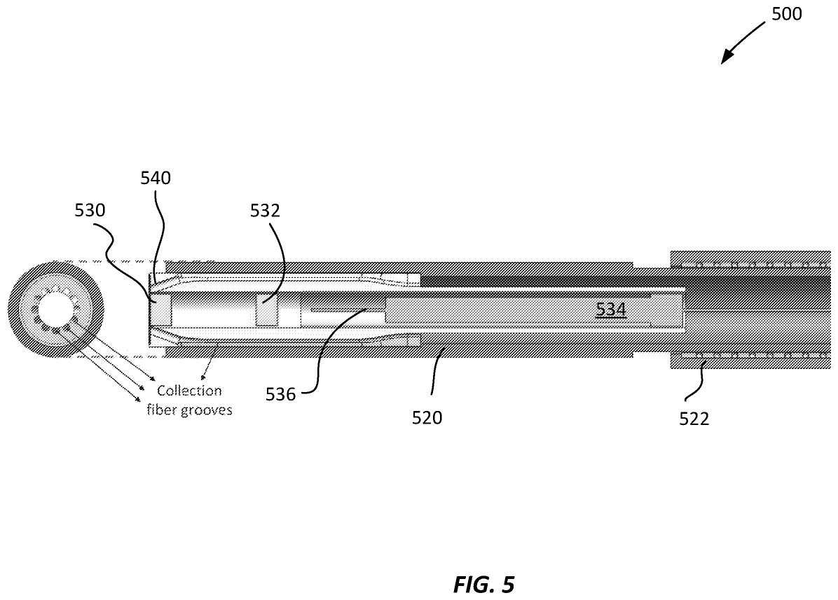

Referring now to FIG. 5, an example side section view of a probe portion 500 of an imaging probe is shown. The probe portion 500 may be similar or identical to the probe portion 402 discussed above in connection with FIG. 4. The probe portion 500 may be fixedly or removably attached to a handle.

As illustrated, the probe portion 500 is a substantially cylindrical body that comprises a plurality of casings/tubings. In the example shown in FIG. 5, the probe portion 500 comprises at least a first casing 520 (e.g., distal casing) and a second casing 522 (e.g., intermediary casing). The first casing 520 defines a distal end of the probe portion 500 and the third casing 524 may be positioned close to a handle of the imaging probe.

As shown, the probe portion 500 comprises at least one actuator 534, at least one laser-guiding element 536, one or more focusing optical elements 530, 532, and at least one signal collecting element 540. Various elements may be at least partially disposed within a housing of the probe portion 500 (e.g., within the probe portion 500 and/or the handle 504).

As depicted, the at least one actuator 534 and at least one laser-guiding element 536 are disposed within the housing/body of the probe portion 500. The at least one actuator 534 can comprise a piezo ceramic tube. The at least one laser-guiding element 536 can comprise a photonic bandgap fiber that extends through the handle and a length of the probe portion 500. The at least one laser-guiding element 536 (e.g., photonic bandgap fiber) can be configured to deliver excitation pulses to a target location, such as living tissue, through at least one excitation optical element (first focusing element 530 and second focusing element 532) via an aperture (e.g., window on a distal surface of the probe portion 500). The at least one laser-guiding element 536 is configured to generate two-photon fluorescence signals, three-photon fluorescence signals, second harmonic generation signals, third harmonic generation signals, and/or the like. The at least one laser-guiding element 536 may be spatially actuated via the at least one actuator 534 (e.g., piezoceramic tube). Excitation pulses can be focused on a target location (e.g., tissue) with an objective fitted on a distal tip (e.g., cap) of the probe portion 500. The probe portion 500 can be actuated to image at adjustable depths into tissue (e.g., up to 2000 μm).

As illustrated in FIG. 5, the probe portion 500/at least one excitation optical element comprises a first focusing element 530 at a distal end of the probe portion 500 and a second focusing element 532. The first focusing element 530 (e.g., first optical lens) is located a first distance from a distal end of the at least one laser-guiding element 536, and the second focusing element 532 (e.g., second optical lens) is located at a second distance from the distal end of the at least one laser-guiding element 536), where the first distance is greater than the second distance.

As further depicted, the probe portion 500 comprises a signal collecting element 540 (e.g., adjacent to the aperture on a distal end of the probe portion 500). The signal collecting element 540 may be operatively coupled with, include, or comprise a spacer component disposed on an exterior portion of the probe portion 500. As shown, the signal collecting element 540 comprises a plurality of fibers (e.g., 12 fibers) circumferentially arranged around the aperture and/or at least a portion of the at least one laser-guiding element 536. Each of the plurality of fibers is cleaved at an angle (e.g., between 1 and 50 degrees) to increase collection directivity and efficiency. In the example shown in FIG. 5, the signal collecting element 540 comprises a ring of fibers. In various implementations, the signal collecting element 540 may comprise multiple rings of fibers (e.g., at least one air-core bandgap fiber or at least one air-core Kagome fiber). The signal collecting element 540 can be configured to receive emitted and/or scattered nonlinearly generated signals without interacting with the at least one excitation optical element (e.g., laser-guiding element 536, first focusing element 530, and second focusing element 532.

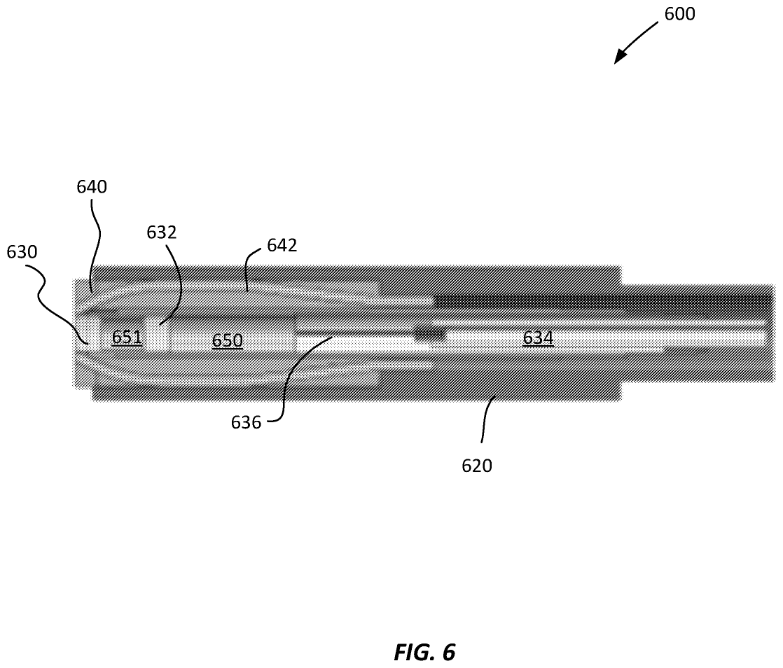

Referring now to FIG. 6, an example side section view of a probe portion 600 of an imaging probe is shown. The probe portion 600 may be similar or identical to the probe portion 302 discussed above in connection with FIG. 3. For example, the probe portion 600 may be fixedly or removably attached to a handle.

As illustrated, the probe portion 600 is a substantially cylindrical body that comprises a plurality of casings/tubings. In the example shown in FIG. 6, the probe portion 600 comprises at least a first casing 620 configured to house one or more optical elements (at least one actuator 634, at least one laser-guiding element 636, one or more focusing optical elements 630, 632, and at least one signal collecting element 640).

As shown, the at least one actuator 634 and the at least one laser-guiding element 636 are at least partially disposed within the housing/body of the probe portion 600. The at least one actuator 634 can comprise a piezo ceramic tube, an SLA printed insert, and the like. The at least one laser-guiding element 636 can comprise a photonic bandgap fiber that extends through the handle and a length of the probe portion 600. The at least one laser-guiding element 636 (e.g., photonic bandgap fiber) can be configured to deliver excitation pulses to a target location, such as living tissue, through at least one excitation optical element (first focusing element 630 and second focusing element 632) via an aperture (e.g., sapphire window). In the example shown in FIG. 6, the probe portion 600 includes a first spacer hypodermic tube 650 and a second spacer hypodermic tube 651. The first spacer hypodermic tube 650 may be configured to set a first distance between a tip of the at least one signal collecting element (e.g., collection fiber(s)) and the objective. The second spacer hypodermic tube 651 may be configured to set a distance between the first focusing lens 630 and the second focusing lens 632.

The at least one laser-guiding element 636 is configured to generate two-photon fluorescence signals, three-photon fluorescence signals, second harmonic generation signals, third harmonic generation signals, and/or the like. The at least one laser-guiding element 636 may be spatially actuated via the at least one actuator 634 (e.g., piezoceramic tube). Excitation pulses can be focused on a target location (e.g., tissue) with an objective fitted on a distal tip of the probe portion 600. The probe portion 600 can be actuated to image at adjustable depths into tissue (e.g., up to 2000 μm).

As illustrated in FIG. 6, the probe portion 600 comprises a first focusing element 630 at a distal end of the probe portion 600 and a second focusing element 632. The first focusing element 630 (e.g., first optical lens) is located a first distance from a distal end of the at least one laser-guiding element 636, and the second focusing element 632 (e.g., second optical lens) is located at a second distance from the distal end of the at least one laser-guiding element 636), where the first distance is greater than the second distance.

As further depicted, the probe portion 600 comprises a signal collecting element 640 (e.g., adjacent to the aperture on a distal end of the probe portion 600). The signal collecting element 640 may be or comprise a spacer component disposed on an exterior portion of the probe portion 600. The signal collecting element 640 may comprise a plurality of fibers circumferentially arranged around the aperture and/or at least a portion of the at least one laser-guiding element 636 (e.g., surrounding the at least one laser guiding element 636). Each of the plurality of fibers is cleaved at an angle (e.g., between 1 and 50 degrees) to increase collection directivity and efficiency. As depicted in FIG. 6, a plurality of fibers, including a first fiber 642 is cleaved at an angle. The signal collecting element 640 can be configured to receive emitted and/or scattered nonlinearly generated signals without interacting with the laser-guiding element 636.

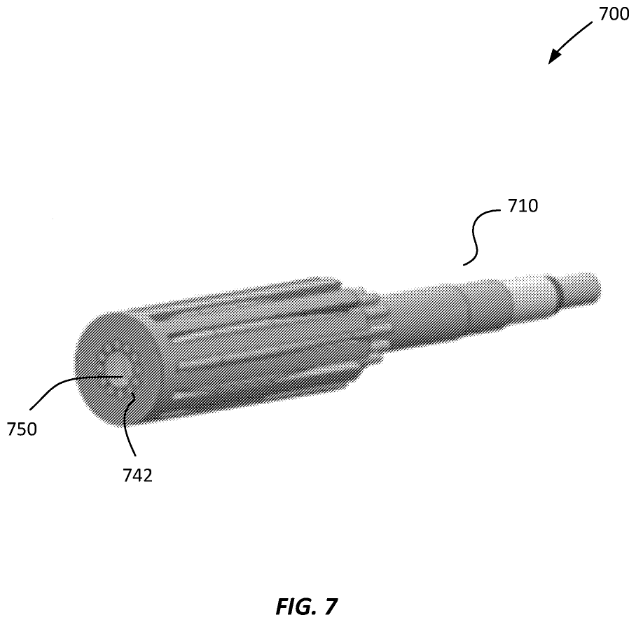

Referring now to FIG. 7, an example perspective view of a signal collecting element 700 is provided. The signal collecting element 700 may be similar or identical to the signal collecting element 640 discussed above in connection with FIG. 6.

The signal collecting element 700 is configured to be operatively coupled to a distal end of an imaging probe 710. In some embodiments, the signal collecting element 700 may comprise any suitable material including plastic, metal, combinations thereof, and the like. As shown, the signal collecting element 700 is embodied as a spacer component disposed/positioned on an exterior portion of the imaging probe 710. In some implementations, where the spacer component is intended for clinical settings, an entirety of the probe may be covered by the spacer component. In some examples, an outer diameter of the spacer component is 14.8 mm, whereas the length of the probe part is 170 mm. In some examples, a distal aperture of the spacer component can be covered with a circular sapphire window (UniversityWafer) of 5.5 mm diameter and 110 μm thickness. In various embodiments, the spacer component may be embodied as a cap, off-axial spacer, cover, protruding member, tip, and the like. The spacer component may be removably attached to or fixedly attached to the probe portion 102. In some examples, the spacer component may cover only a portion of a distal end of the probe portion 102.

As illustrated, the signal collecting element 700 comprise a ring of fibers circumferentially arranged around an aperture 750 on a surface of the imaging probe 710 and the at least one laser-guiding element positioned within the imaging probe 710. In some examples, the signal collecting element 700 comprises a plurality of fibers, for example, multiple rings or layers of fibers (e.g., air-core bandgap fibers, air-core Kagome fibers, and/or the like). In some embodiments, each of the plurality of fibers may be at least partially disposed within a separate channel (e.g., tube, tubular member or the like). In some embodiments, a bundle of fibers may be disposed within the same channel. Each of the plurality of fibers (e.g., first fiber 742) is cleaved at an angle (e.g., between 1 and 50 degrees), bowed, bent, slanted, or the like to increase collection directivity and efficiency. The use of slanted-cut fibers in a bent arrangement increases collection directivity and efficiency, as discussed in more detail herein. Use of multiple collection fibers increases effective collection area, further increasing collection efficiency. Simulations indicate that increase in collection efficiency is more prevalent within media having scattering lengths around 100 μm, which is the case for human epithelial tissues. The signal collecting element 700 (e.g., cap) can be configured to receive emitted and/or scattered nonlinearly generated signals without interacting with other optical elements in the imaging probe 710.

Referring now to FIG. 8A and FIG. 8B, schematic diagrams depicting an axial scanning operation in relation to an imaging probe 800A and 800B are provided. The imaging probe may be similar or identical to the imaging probe 710 including signal collecting element 700 discussed above in connection with FIG. 7.

As depicted in FIGS. 8A and 8B, a change of imaging depth is denoted as “ID” with axial scanning, where ID is understood as the distance from the spacer component aperture 850A and 850B to the focal spot position. Since the position of the spacer component aperture and the working distance (denoted as “WD”) of the probe objective are fixed quantities, axial actuation of the imaging probe 800A and 800B causes the focal spot to move with respect to the spacer component 860A and 860B. Triangles 820A and 820B represent laser focusing, whose dimensions were exaggerated for clarity.

Experimental Results

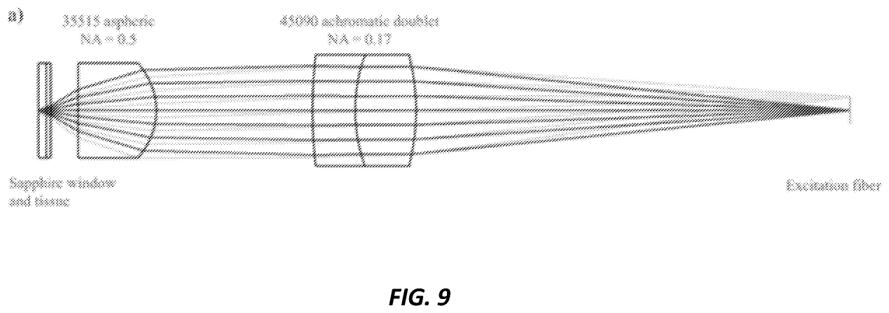

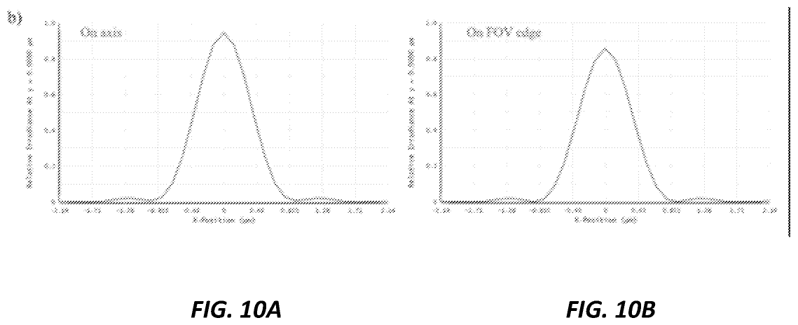

Referring now to FIG. 9, a schematic diagram depicting an example miniaturized objective is provided. Design of the miniaturized objective was done in Zemax environment. Measured output divergence of the fiber was used in the model and several designs consisting of commercially available lenses with 3 mm diameters were investigated. Finalized design after optimization is shown in FIG. 9. The design consists of a 0.17 NA achromatic doublet lens (Edmund Optics, #45-090) to collimate the laser beams coming out of the excitation fiber and a 0.5 NA aspheric lens (Lightpath, 355151), to focus the beam on the output. The model considers the presence of the sapphire window in the spacer component aperture. Simulation results suggest a 1/e2 IPSF diameter of 1.34 μm. According to the models presented, this corresponds to lateral and axial IPSF2 of 0.56 μm and 3.33 μm, respectively. Rated clear aperture of the lenses set the maximum FOV of this system at 128 μm, for which requires the excitation fiber tip to deflect 330 μm from the axis. FIG. 10A and FIG. 10B are graphs depicting Strehl ration profiles for on-axis and off-axis focus, respectively. With reference to FIG. 10A and FIG. 10B, at the FOV edge, Strehl ratio drops to 0.86 from its on-axis value of 0.94. Working distance of the miniaturized objective is around 1 mm.

While assembling the miniaturized objective, the lenses were placed in the same hypodermic tubing. Design distance between the lenses was set with an SLA printed thin sheath (depicted in FIG. 6) that conformally wraps inside the hypodermic tube. Miniaturized objective design includes the distance between the excitation fiber and the objective itself. This distance is set with a separate segment of hypodermic tubing (second spacer hypodermic tube 651 shown positioned between the first focusing optical element 630 and the second focusing optical element 632 in FIG. 6), which was cut to the length dictated by the design within a tolerance of 50 μm.

All three hypodermic tube segments were then placed inside a larger hypodermic tube (MicroGroup, 304H9XX) with an inner diameter matching the outer diameters of the smaller hypodermic tubes. The complete excitation unit was then inserted into the fiber holder part (brass tubing 320 depicted in FIG. 3). This part was incorporated in the design to position and align the collection fibers and the excitation unit securely at the distal tip of the first tubing.

Collection Fibers and Collection Optics

Autofluorescence collection is handled by 12 multimode collection fibers with 0.5 NA and 735 μm core diameter (Edmund Optics, #02-533, shown as first fiber 642 in FIG. 6). These fibers are radially arranged around the excitation optics. The collection approach implemented here increases the available collection area to 20 mm2, 100-fold from what DCFs offer. A drawback of this approach is that individual collection elements being positioned off-axis. To compensate for this, the collection system was designed such that the collection fibers arrive at the distal tip with a finite bend angle and are similarly cleaved at that angle. The angular cleaving of the collection fibers compensates for the off-axis positioning, since such termination increases the directivity of collection towards the probe axis. Increase of collection efficiency with the increase of the bend/termination angle can be demonstrated with Zemax simulations.

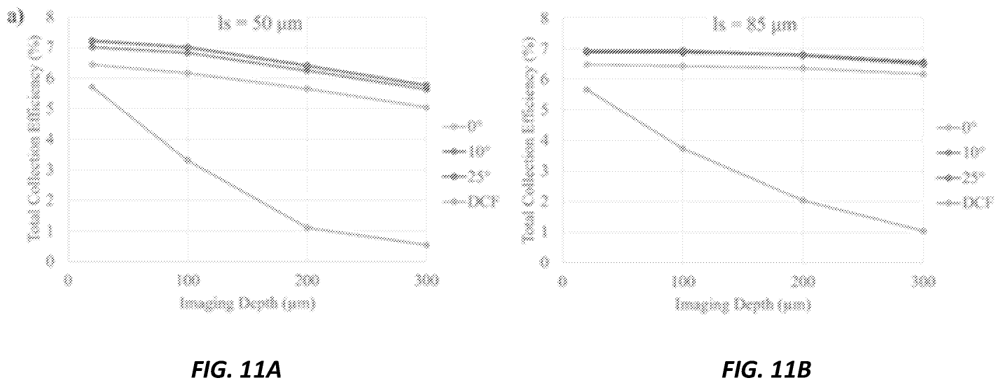

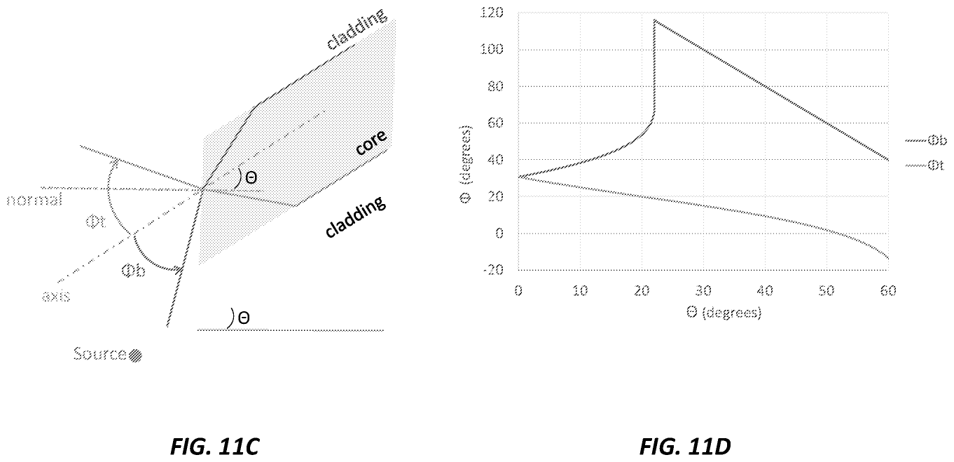

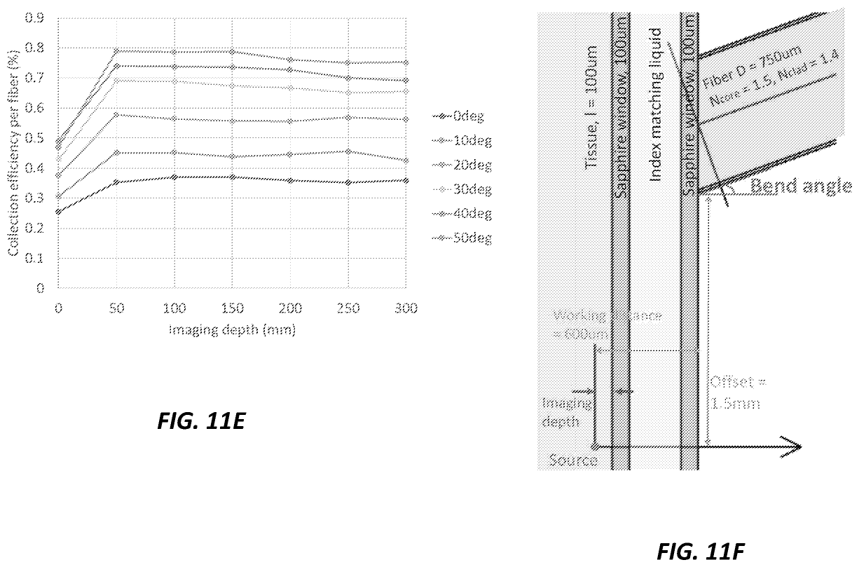

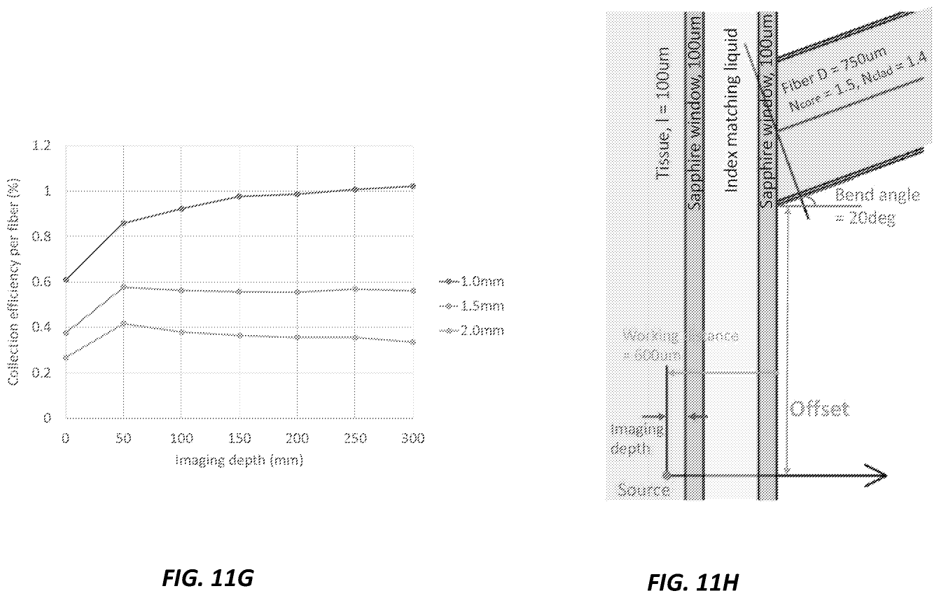

FIGS. 11A-J are schematic diagrams depicting various simulation results. In FIGS. 11C-J, the simulations assume a second sapphire window is present at the tip of the objective/collection fibers and that there exists an index matching liquid between two sapphire windows. In the simulations presented in FIGS. 11A-B, the second window and the index matching liquid are not present. In the simulations for FIG. 11A and FIG. 11B, the excited fluorescence volume is modeled at as a point source, present in a medium that scatters according to Henyey-Greenstein model with g=0.9. The position of the point source, which corresponds to different imaging depths, and the mean free path (1 s) of the scattering medium are adjusted to obtain the results presented in FIG. 11A and FIG. 11B. In particular, FIG. 11A and FIG. 11B are graphs depicting a change in collection efficiency at different imaging depths and mean free paths, when the example imaging probe uses the proposed collection approach at different bend/cleaving angles as well as the case of a DCF These simulations also considered in various scenarios, where embodiments of the present disclosure use the DCF with large silica cladding of 300 μm diameter for fluorescence collection.

FIG. 11C and FIG. 11D are a schematic diagram and graph, respectively, depicting simulation results for the effect of cleaving. FIG. 11C and FIG. 11D demonstrate that cleaving fiber tips at an angle increases directivity of individual fibers.

FIG. 11E and FIG. 11F are a graph and schematic diagram, respectively, depicting simulation results for the effect of angle on collection. FIG. 11E and FIG. 11F demonstrate that collection efficiency increases as bend angle increases.

FIG. 11G and FIG. 11H are a graph and schematic diagram, respectively, depicting simulation results for the effect of offset distance on collection. As demonstrated in FIG. 11G and FIG. 11H, collection efficiency increases as the offset distance decreases.

FIG. 11I and FIG. 11J are a graph and schematic diagram, respectively, depicting simulation results for the effect of scattering on collection. As demonstrated in FIG. 11I and FIG. 11J, the highest collection efficiency was obtained with a mean free path of 100 μm which shows that embodiments of the present disclosure perform optimally with respect to scattering. In particular, the collection efficiency does not change drastically with changes in scattering, and scattering related imaging depth limitations are compensated for.

Simulations confirm that increasing the bend and cleaving angle increases the collection efficiency with diminishing returns. For embodiments of the imaging probe described herein, the presented fluorescence collection outperforms the DCF collection, particularly at higher imaging depths. This suggests that the presented fluorescence collection approach is useful particularly in high scattering settings. Collection efficiency limitations at increased imaging depths with DCF collection is noted in conventional systems, where a 3.3% simulated collection efficiency can be obtained with a DCF with cladding diameter of 100 μm. Radial symmetry of the presented collection approach compensates for any deviations from the axial position of the focal spot. According to simulations, scanning of the focal spot will not result in any change in collection efficiency. On the other hand, for the DCF scenario, simulations indicate collection efficiency reductions up to 28% when the focal spot is placed at the FOV edge.

Whereas increased bending/cleaving angles seem to marginally increase the collection efficiency, they contribute to increase in probe diameter and introduce the risk of losses due to sharper fiber bends. As a result, a bend and cleaving angle of 25° was used, which offers a significant increase in collection efficiency at minimal bending losses of, according to obtained measurements. To ensure additional losses remain minimal, grooves around the fiber holder part were designed to allow the collection fibers to realign with the probe tubings at a more lenient angle.

Collecting signals with a multitude of fibers introduces a challenge in fiber to sensor coupling since the total diameter of the collection fiber bundle becomes comparable to the sensor diameter of 5 mm for the selected PMT (Hamamatsu, H7422PA-40). In the example of twelve fibers, arrangement of the most compact bundle results in three concentric layers of fibers, as depicted in FIG. 11B. Design of the dedicated collection optics system was based on the optimization of overall coupling efficiency, which was simulated to be 65% according to Zemax simulations. During assembly, the collection fiber bundle was kept in the shown arrangement with an SLA printed piece.

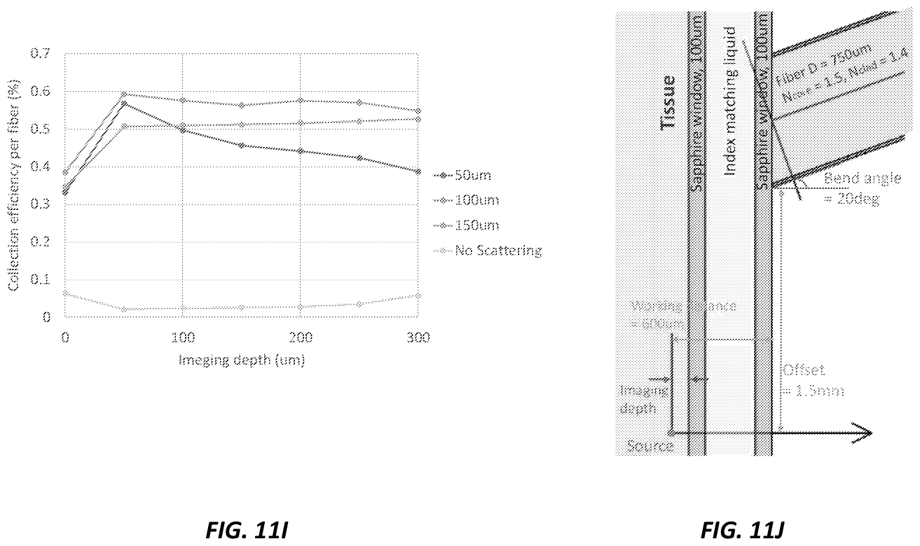

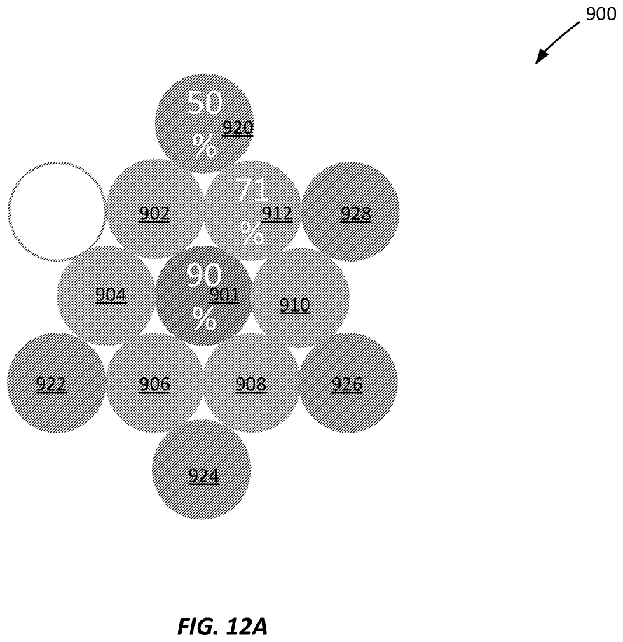

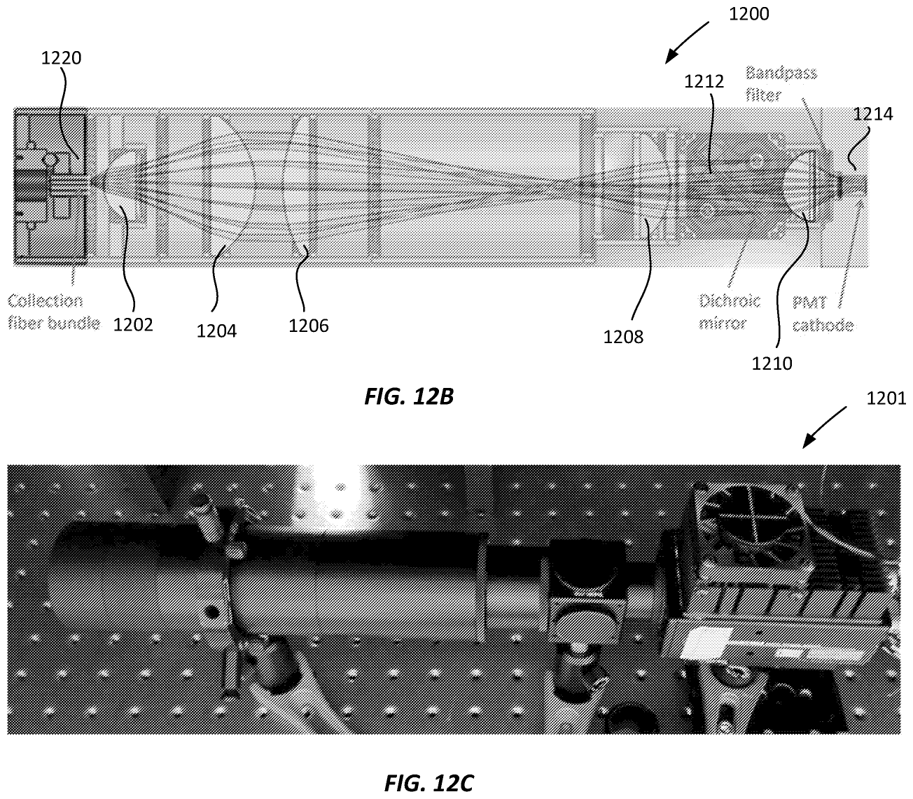

Referring now to FIG. 12A, a schematic diagram depicting an example collection fiber bundle (also referred to herein as a signal collecting element comprising a plurality of fibers) is provided. In some embodiments, high NA and high overall diameter of collection fiber bundle requires the use of a dedicated optical system for efficient coupling of the bundle to one or more PMT modules.

As shown, the collection fiber bundle comprises twelve optical fibers that can be arranged at an input of the collection optics system in three layers. A first fiber 910 defines a first layer; a first set of fibers 902, 904, 906, 908, 910, 912 defines a second layer; and a second set of fibers 920, 922, 924, 926, 928 defines a third layer of the bundle arrangement.

Transmission efficiency of an individual fiber depends on its location within the bundle. In the example shown in FIG. 12A, the first layer has a transmission efficiency of 90%; the second layer has a transmission efficiency of 71%, and the third layer has a transmission efficiency of 60%. In some simulations, overall efficiency was 64% based on simulations. Experimentally, it was measured to be approximately 61% in some examples.

Using 12 high NA fibers for fluorescence collection requires careful design of a collection optics system to efficiently couple the fluorescence signals from all fibers to the PMT. Since the NA of the collection fibers are high and the overall diameter of the collection fiber bundle is comparable to conventional PMT detectors, high power spherical lenses may be used to collimate the beams exiting the collection fibers (e.g., signal collecting element). Spherical aberrations are not the main concern here, since the aim is not necessarily to image the bundle on the PMT surface, but rather to increase the coupling efficiency. Since the collimated beams exiting the collection fibers have a diameter higher than that of the conventional 1-inch size filter and mirror systems, additional lenses were added to reduce a diameter of the beam (e.g., 1-inch or lower). A dichroic mirror can be added to this optical system to enable working at multiple nonlinear imaging modes that may require collecting signal at multiple different emission wavelengths simultaneously. A standard size dichroic mirror can be used for fluorescence signal separation at different wavelengths. For this purpose, a collection optics system was designed and assembled formed of 5 spherical lenses of diameters of 1″, 1.5″, and 2″. Design of the collection optics was done using Zemax optics software. It was observed that collection fibers forming the fiber bundle achieve different coupling efficiencies, depending on where they are located on the bundle. As depicted in FIG. 12A, 12 fibers can be placed tightly in a bundle, forming 3 concentric layers. From center to the periphery, fibers on different layers can achieve coupling efficiencies of 90%, 71%, and 50%, while considering the aperture limitations on the front part of the PMT detector. These numbers correspond to an overall coupling efficiency of 65%.

Referring now to FIG. 12B and FIG. 12C, a schematic diagram depicting example collection optics systems 1200 and 1201 are provided. As shown, the finalized design comprises optomechanical tubing components that house various optical elements. In various embodiments, the collection optics system 1200 comprises a plurality of optical elements, such as lenses, mirror, and the like. In particular, FIG. 12B shows a Zemax design of collection optics superimposed with a technical drawing of optomechanical tubings that house them. In the example shown in FIG. 12B, the collection optics system 1200 comprises a first lens 1202 positioned adjacent to a collection fiber bundle 1220 (e.g., signal collecting element), a second lens 1204, a third lens 1206, a fourth lens 1208, and a fifth lens 1210 positioned adjacent to a PMT 1214. As further depicted, a dichroic mirror 1212 is positioned between the fourth lens 1208 and the fifth lens 1210. During the assembly, spacing between the lenses 1202, 1204, 1206, 1208, and 1210 was done by 3D printed brace parts placed inside the optomechanical tubings. These brace parts are thin enough to not interfere with the clear apertures. An SLA printed cylindrical element was designed to hold the collection fiber bundle together at the input of the collection optics. Outer diameter of this piece matches the inner diameter of an optical fiber holder piece, which offers tilt and yaw adjustment for coupling optimization. Fiber holder piece and the PMT aperture can easily be integrated with the rest of the collection optics tubing, forming a fully enclosed system 1201 as depicted in FIG. 12C (e.g., identical to the collection optics system 1200 depicted in FIG. 12B).

Setup and Read-Out

Parts of the probe part of the endoscope that are directly involved in axial scanning were fabricated out of stainless steel. For the prototype used in benchtop tests, fabrication using SLA printing of the spacer component and the handle were utilized for their large size and the fiber holder for its complexity. In a clinical application, it will be required to fabricate the spacer component out of a biocompatible and durable material, such as stainless steel. Excitation fiber and collection fiber bundle lengths were left at 6 m and 2 m, respectively. Exposed parts of the fibers were isolated and protected with furcation tubing (ThorLabs, FT020 and FTS061A).

The assembled probe was mounted on a benchtop structure with translation capability in three dimensions for the laboratory tests. Excitation fiber was coupled to a Ti:sapphire laser (Spectra Physics, MaiTai) with a tunable wavelength, 100 fs pulse duration, and 80 MHz repetition rate. The laser wavelength was set to 775 nm for the subsequent experiments. Reflected laser signals were filtered in the collection optics with a bandpass filter (Schott, BG39).

The piezo actuator tube was driven in a fully differential fashion, using a programmable function generator (Rigol Technologies, DG2052), whose output was amplified with custom-design amplifiers with voltage gains of 10. Resonant scanning of the fiber tip with the piezo actuator is prone to distortions when operating close to the resonance frequency of the fiber overhang. Conventional methods were used to minimize these distortions when designing the spiral scan signals at 1.1 kHz for high fiber deflection. Since the piezo actuator tube does not provide position feedback and due to nonlinear effects involved in the resonant scanning, a straightforward estimation of the actual scan trajectory from the applied signals is hard to attain. Instead, a position sensing detector (Thorlabs, PDP90A) was used to calibrate the designed scan trajectories and test their repeatability as presented previously 10. Data obtained during calibration is needed for correct signal to pixel assignment during imaging and is stable over a day.

A low noise amplifier (Stanford Research Systems, SR570) was used for interfacing PMT signals with a 1.25 MHz data acquisition card (National Instruments, NI 6356). A custom design code was developed in Matlab to coordinate the programming of the function generator, data acquisition, lateral scan calibration, axial actuation, and generation of images.

Results

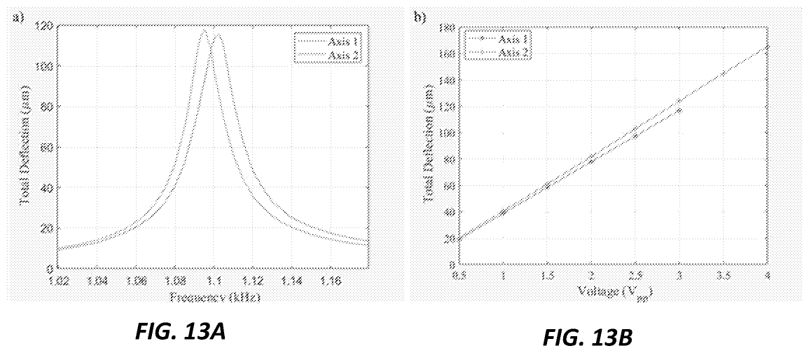

Assembled fiber scanner and miniaturized objective were used as a single unit for the characterization of the lateral scanning capability. Position sensing detector was used for the tracking of the focal spot, which was focused through the miniaturized objective and the sapphire window on the spacer component aperture. Deflection of the focal spot was measured at different actuation frequencies and amplitudes. Results are presented in FIG. 13A and FIG. 13B which are graphs depicting measured deflection characteristics of the focal spot. Total deflection refers to the distance between two extreme positions of the focal spot trajectory, which is an indication of the potential FOV. FIG. 13A depicts total deflection of the focal spot for either axis vs. actuation signal frequency. Actuation signal amplitude was set at 3 V at the function generator and was amplified by a factor of 10 by the voltage amplifiers. FIG. 13B depicts total deflection of the focal spot for either axis vs. actuation signal amplitude. X-axis values show the applied voltage before the voltage amplifiers of gain 10. Both axes were actuated at their respective resonance frequencies.

Electrode axes of the piezo actuator tube were arbitrarily but consistently labeled as axis 1 and axis 2. Change of beam deflection with actuation signal frequency follows the pattern of a mechanical resonator as expected. Both axes express resonance frequencies that are similar but not the same. This is due to radial asymmetries in the mechanical systems and is one of the prominent nonlinearities causing the deformations in the spiral scan that needs to be corrected. It is seen that for both axes, the relationship between the focal spot deflection amplitude and the actuation signal amplitude is very linear as expected. In our subsequent imaging operations, we set the actuation frequency at 1.1 kHz to maximize deflection in both axes, which helps achieve the FOV limit while driving the piezo signals at voltage amplitudes below 30 Volts (V).

With the spacer component length used in the experiments, the probe remains retracted from the desired axial position by 3.2 mm. This design choice was done so that the optical surfaces at the distal tip can be better protected. In other words, probe operation requires an initial translation of the probe tip in distal direction, which compresses the spring in the probe part further during imaging. High compression of spring contributes to motor actuation stability. In the described conditions, we measured the hysteresis of axial scan movement to be smaller than 5 μm.

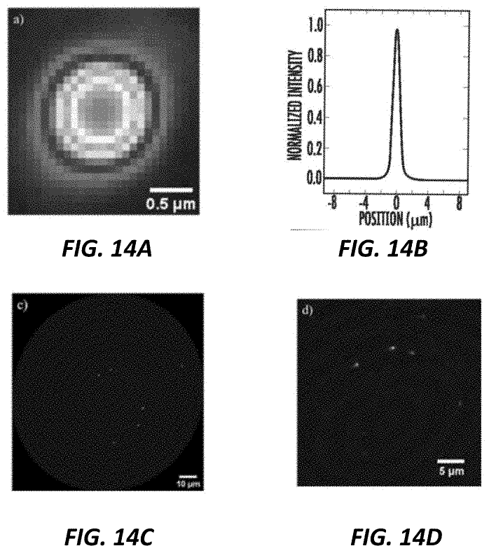

The shape of the focal spot was characterized using a beam profiler (Ophir, SP932U), in the presence of the sapphire window in the spacer component aperture. A gaussian function was fit to the cross-sections of the intensity map obtained by the beam profiler with R2 of 0.99. Results are shown in FIG. 14A, FIG. 14B, FIG. 14C, and FIG. 14D. FIG. 14A depicts an intensity image of the focal spot obtained by the beam profiler. FIG. 14B depicts a Gaussian fit (R2=0.99) to the beam profile obtained from the intensity image. FIG. 14C depicts an image of a single layer of 100 nm diameter fluorescent beads obtained with the probe. Pixel size and FOV are 0.33 μm and 120 μm, respectively. FIG. 14D depicts a close-up of the central portion of the image presented in FIG. 14C. Bead images were used in real resolution characterization.

The fit function yields a 1/e2 diameter for IPSF of 1.62±0.2 μm. This number corresponds to a lateral and axial FWHM diameter for IPSF2 of 0.67±0.1 μm and 4.92±0.1 μm, which is a theoretical estimate of imaging resolution. Lateral FWHM diameter for IPSF2 is about 10% larger than what simulated characteristics of the miniaturized objective offered. This can be attributed to the small errors in objective assembly, which can have a visible effect on a high NA system.

For the measurement of real optical resolution, the probe was used to image a single layer of fluorescent beads of 100 nm diameter (ThermoFisher, F8803) at axial positions separated by 0.5 μm. A pixel size of 0.33 μm, half of expected lateral resolution, was chosen for image construction. Image from one of the regions of interest is shown in FIG. 14C, and FIG. 14D. Average FWHM diameter of intensity profiles from bead images were used to measure real lateral and axial imaging resolution, which were found to be 0.64 μm and 4.10 μm, respectively. These values are close to what we expected from the focal spot size characterizations.

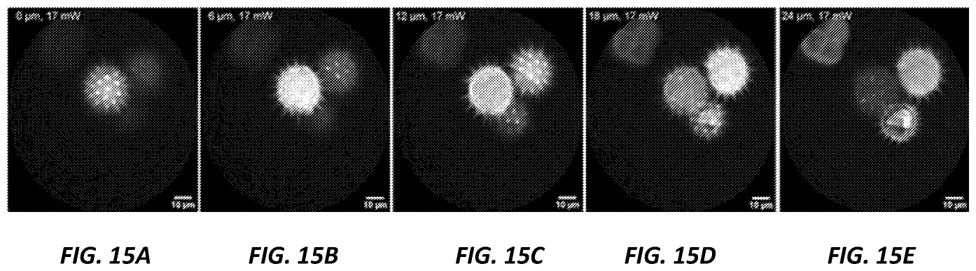

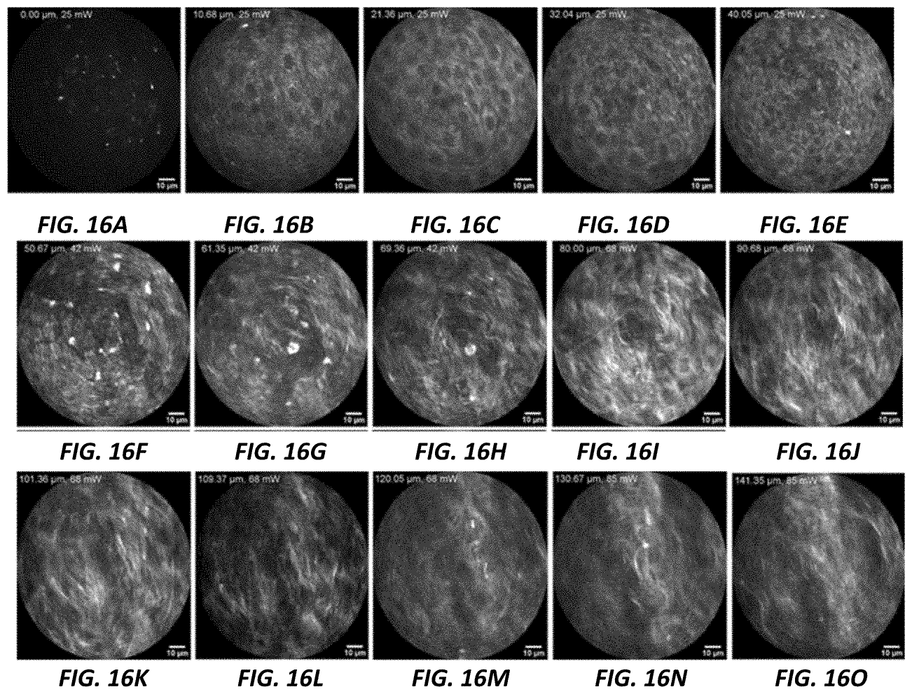

Following the characterization experiments, an imaging probe was used to image biological samples. A slide of mixed pollen cores (Carolina Biological Supply, #304262) was initially used as a reference sample. FIG. 15A, FIG. 15B, FIG. 15C, FIG. 15D, and FIG. 15E show images of a pollen core cluster obtained with the imaging probe obtained at different imaging depths/different axial positions from a region of interest within a sample slide showing a pollen core cluster. Axial positions and average power at the sample are shown on respective images. Averages of 10 scans per axial position are shown at a scan period of 0.5 s. Pixel size and FOV are 0.33 μm and 120 μm, respectively. Average laser power at the sample was 17 mW, at a scanning period of 0.5 s. Images were formed by averaging 10 frames. No additional filters or nonlinear contrast enhancement algorithms were applied. Images at different depths were obtained with an automated axial scan procedure. FIG. 16A, FIG. 16B, FIG. 16C, FIG. 16D, FIG. 16E, FIG. 16F, FIG. 16G, FIG. 16H, FIG. 16I, FIG. 16J, FIG. 16K, FIG. 16L, FIG. 16M, FIG. 16N, and FIG. 16O are schematic diagrams depicting results of various simulations.

Finally, the probe was used to perform autofluorescence imaging of freshly excised porcine vocal fold tissues. Samples were imaged approximately 1 hour after harvesting, during which they were kept in ice. The same scanning and image construction parameters as before were used, while increasing the imaging depth and adjusting the average power at sample surface accordingly. Fluorescent beads were applied on tissue for easier detection of the surface. Obtained images are shown in FIGS. 16A-160. In particular, FIGS. 16A-160 shows autofluorescence images obtained with the probe at different imaging depths into a freshly excised porcine vocal fold tissue sample. Imaging depths and average power at the sample are shown on respective images. First image shows fluorescent beads applied on tissue for detection of surface. Averages of 10 scans per axial position are shown at a scan period of 0.5 s. Pixel size and FOV are 0.33 μm and 120 μm, respectively. Similarly, no additional filters or nonlinear contrast enhancement algorithms were applied.

### CONCLUSION

A 2p autofluorescence imaging endoscope system intended for early detection of cervical cancer was designed, assembled, and characterized. One of the prominent features of our design was the collection approach, where the overall collection efficiency was improved by increasing the effective collection area and directivity of collection. Simulation results show that this approach is advantageous, particularly at high imaging depths and in high scattering media. The fact that the collection fibers do not interface with any additional optics simplify the design and assembly of the endoscope system significantly. Availability of high precision three-dimensional (3D) printing technologies further simplify realization of such a fluorescence collection system. Further variations on this approach may increase the collection efficiency even further in future iterations. It should be noted that since this collection approach is based on collection fibers positioned off-axis, it is more efficient with systems offering high working distances. This is advantageous in our case, since the way axial scanning is implemented also calls for a design offering a working distance that is not very short.

This being said, the working distance cannot be kept too long due to the trade-off between the working distance and resolution Aiming to detect cancer at early stages, which causes subtle and small changes, the endoscope system needs to achieve cellular level resolution. Characterization results indicate that the endoscope system, based on a miniaturized objective consisting of two commercially available lenses, was able to achieve a lateral and axial resolution equivalent to an optical system with NA of 0.44. While this level of resolution can be satisfactory for metabolic imaging, higher performance can be obtained with dedicated custom design optics. Such design can also improve FOV, which would increase diagnosis sensitivity.

Example Computing Device

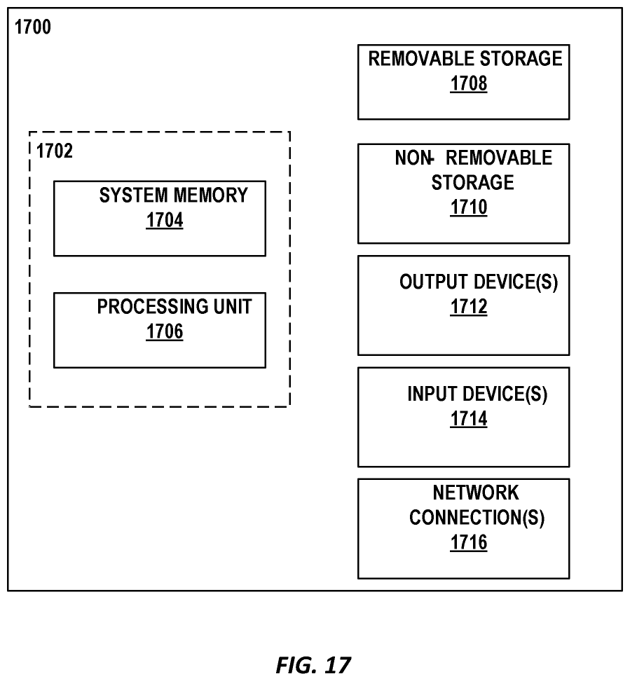

It should be appreciated that the logical operations described herein with respect to the various figures may be implemented (1) as a sequence of computer implemented acts or program modules (i.e., software) running on a computing device (e.g., the computing device described in FIG. 17), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 17, an example computing device 1700 upon which embodiments of the invention may be implemented is illustrated. This disclosure contemplates that the controller(s) for operating the flexure elements and/or imaging apparatus can be implemented using computing device 1700. It should be understood that the example computing device 1700 is only one example of a suitable computing environment upon which embodiments of the invention may be implemented. Optionally, the computing device 1700 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media. In some embodiments, the computing device 1700 comprises or is operatively coupled to a detector component and/or an optical system, such as, but not limited to one or more PMTs, one or more Hybrid (HyD) detectors, and the like.

In its most basic configuration, computing device 1700 typically includes at least one processing unit 1706 and system memory 1704. Depending on the exact configuration and type of computing device, system memory 1704 may be volatile (such as random access memory (RAM)), non-volatile (such as read-only memory (ROM), flash memory, etc.), or some combination of the two. This most basic configuration is illustrated in FIG. 17 by dashed line 1702. The processing unit 1706 may be a standard programmable processor that performs arithmetic and logic operations necessary for operation of the computing device 1700. The computing device 1700 may also include a bus or other communication mechanism for communicating information among various components of the computing device 1700.

Computing device 1700 may have additional features/functionality. For example, computing device 1700 may include additional storage such as removable storage 1708 and non-removable storage 1710 including, but not limited to, magnetic or optical disks or tapes. Computing device 1700 may also contain network connection(s) 1716 that allow the device to communicate with other devices. Computing device 1700 may also have input device(s) 1714 such as a keyboard, mouse, touch screen, etc. Output device(s) 1712 such as a display, speakers, printer, etc. may also be included. The additional devices may be connected to the bus in order to facilitate communication of data among the components of the computing device 1700. All these devices are well known in the art and need not be discussed at length here.

The processing unit 1706 may be configured to execute program code encoded in tangible, computer-readable media. Tangible, computer-readable media refers to any media that is capable of providing data that causes the computing device 1700 (i.e., a machine) to operate in a particular fashion. Various computer-readable media may be utilized to provide instructions to the processing unit 1706 for execution. Example tangible, computer-readable media may include, but is not limited to, volatile media, non-volatile media, removable media and non-removable media implemented in any method or technology for storage of information such as computer readable instructions, data structures, program modules or other data. System memory 1704, removable storage 1708, and non-removable storage 1710 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1706 may execute program code stored in the system memory 1704. For example, the bus may carry data to the system memory 1704, from which the processing unit 1706 receives and executes instructions. The data received by the system memory 1704 may optionally be stored on the removable storage 1708 or the non-removable storage 1710 before or after execution by the processing unit 1706.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. An imaging probe comprising:
a housing;
at least one excitation optical element at least partially disposed within the housing;
the at least one excitation optical element comprising at least one laser-guiding element being configured to deliver excitation pulses to a target location through the at least one excitation optical element via an aperture; and
a signal collecting element disposed adjacent to the at least one excitation optical element.

2. The imaging probe of claim 1, wherein the signal collecting element is configured to receive the emitted and/or scattered nonlinearly generated signals without interacting with the at least one excitation optical element.

3. The imaging probe of claim 2, wherein the emitted and/or scattered nonlinearly generated signals comprise at least one of two-photon fluorescence signals, three-photon fluorescence signals, second harmonic generation signals, and third harmonic generation signals.

4. The imaging probe of claim 1, wherein the imaging probe is operatively coupled to a spacer component that is configured to be positioned adjacent to the signal collecting element, and wherein the spacer component is configured to create a space between the signal collecting element and a tissue.

5. The imaging probe of claim 1, wherein the signal collecting element comprises a plurality of fibers circumferentially arranged around at least a portion of the at least one laser-guiding element.

6. The imaging probe of claim 5, wherein the plurality of fibers comprises a number between three and forty fibers.

7. The imaging probe of claim 5, wherein each of the plurality of fibers is cleaved at an angle.

8. The imaging probe of claim 7, wherein the angle is between 1 degree and 50 degrees.

9. The imaging probe of claim 1, wherein the signal collecting element comprises multiple rings of fibers.

10. The imaging probe of claim 1, wherein the laser-guiding element comprises at least one fiber extending through at least a portion of the housing.

11. The imaging probe of claim 10, wherein the at least one fiber comprises an air-core bandgap fiber or an air-core Kagome fiber.

12. The imaging probe of claim 1, wherein the aperture comprises a transparent window.

13. The imaging probe of claim 1, wherein the at least one excitation optical element comprises a plurality of focusing lenses.

14. The imaging probe of claim 2, further comprising a detector component operatively coupled to the imaging probe that is configured to receive the emitted and/or scattered nonlinearly generated signals and output image data via a display.

15. The imaging probe of claim 13, wherein the detector component comprises at least one of a photomultiplier-tube (PMT) module and a Hybrid (HyD) detector.

16. The imaging probe of claim 1, wherein the imaging probe is embodied as an endoscope or table-top nonlinear microscope.

17. The imaging probe of claim 1, further comprising a handle portion, wherein the at least one laser-guiding element and at least a portion of the signal collecting element extends through the handle portion and a length of the housing.

18. The imaging probe of claim 17, further comprising a motor disposed within the handle portion.

19. The imaging probe of claim 18, wherein the motor is configured to actuate axial scanning of the imaging probe to facilitate an imaging depth adjustment.

20. A system comprising:
an imaging probe, the imaging probe comprising:
a housing,
at least one excitation optical element at least partially disposed within the housing,
the at least one excitation optical element comprising at least one laser-guiding element being configured to deliver excitation pulses to a target location through the at least one excitation optical element via an aperture, and
a signal collecting element adjacent to the at least one excitation optical element; and
a detector component operatively coupled to the imaging probe that is configured to receive emitted and/or scattered nonlinearly generated signals via the signal collecting element and output image data via a display.

21. The system of claim 20, wherein the signal collecting element is configured to receive the emitted and/or scattered nonlinearly generated signals without interacting with at least one of the at least one excitation optical element while interacting with at least one focusing optical element external to the imaging probe.

22. The system of claim 20, wherein the imaging probe has an imaging signal depth between 0-2000 μm.
